(19) United States
(12) Patent Application Publication
(10) Pub. No.: US 2022/0257562 A1
(43) Pub. Date: Aug. 18, 2022

(54) METHOD FOR IMPROVING ENDOMETRIOSIS AND METHOD FOR PRODUCING A MATERIAL USED THEREFOR

(71) Applicants: NICHIMO CO., LTD., Tokyo (JP); KYOTO PREFECTURAL PUBLIC UNIVERSITY CORP., Kyoto-shi (JP)

(72) Inventors: Tomohiro AMAMI, Tokyo (JP); Soichiro SHIGEHIRO, Tokyo (JP); Jo KITAWAKI, Kamigyo-ku (JP); Osamu TAKAOKA, Kamigyo-ku (JP)

(21) Appl. No.: 17/738,752

(22) Filed: May 6, 2022

Related U.S. Application Data
(62) Division of application No. 16/489,321, filed on Jan. 17, 2020, now abandoned.

(30) Foreign Application Priority Data
Feb. 28, 2017 (JP) 2017-036544

(51) Int. Cl. A61K 31/352; A61P 15/00

(52) U.S. Cl. CPC A61K 31/352; A61K 2236/19; A61P 15/00

(57) ABSTRACT

A method for improving endometriosis includes administrating a composition containing aglycon type isoflavones and a pharmaceutically acceptable carrier to mammal subject in need of treatment of endometriosis in an amount effective for treating endometriosis, wherein the aglycon type isoflavones are composed of daidzein, genistein, and glycitein.

### CROSS-REFERENCE TO RELATED APPLICATIONS

This application is a divisional of U.S. patent application No. 16/489,321, filed Jan. 17, 2020 (371(c) Date), which is the U.S. National Phase under 35 U.S.C. § 371 of International Application PCT/JP2018/007328, filed Feb. 27, 2018, which claims priority to Japanese Patent Application No. JP2017-036544, filed Feb. 28, 2017, each disclosure of which is herein incorporated by reference in its entirety. The International Application was published under PCT Article 21(2) in a language other than English. The applicants herein explicitly rescind and retract any prior disclaimers or disavowals or any amendment/statement otherwise limiting claim scope made in any parent, child or related prosecution history with regard to any subject matter supported by the present application.

### TECHNICAL FIELD

The present invention relates to a material for improving endometriosis, and more particularly to a material for improving endometriosis using aglycone type isoflavone (also referred to as “isoflavone aglycone”) and a method for producing the same.

### BACKGROUND ART

In general, it is desired to improve endometriosis, and, for example, therapeutic agents for endometriosis-related diseases (see Patent Literature 1) and the like are also proposed. However, the therapeutic agent described in Patent Literature 1 is not to be treated in response to estrogen which many women possess.

On the other hand, endometriosis is a disease in which an endometrium-like tissue engrafts out of the uterine cavity and spreads, and affects 5% to 10% of women of reproductive age (Non-patent Literature 1). The main symptoms of endometriosis are pelvic pain and infertility (Non-patent Literature 2). It is well known that estrogen is involved in the development and maintenance of lesions since menopause improves symptoms (Non-patent Literature 3). At the endometriotic lesion local site, estrogen directly contributes to inflammation by inducing cytokines (Non-patent Literature 4). Inflammatory cytokines promote estrogen synthesis locally at endometriotic lesions, and as a result, positive feedback promotes the development and maintenance of the lesions (Non-patent Literature 5). Thus, endometriosis is considered as an estrogen-dependent inflammatory disease, and both hormones and inflammatory cytokines are involved in the development, maintenance and clinical symptoms of lesions (Non-patent Literature 6).

Endocrine treatments for endometriosis, such as low-dose estrogen-progestin, GnRH agonists, progestin, etc., are effective for focal development and pelvic pain. However, these can not be used for women who wish to become pregnant immediately because they suppress ovulation. There is a need for new treatments for endometriosis that can be used in all endometriosis patients with fewer side effects.

Isoflavones are non-steroidal natural products derived from plants with weak estrogenic activity. Since isoflavones are similar in structure to estrogens, they can bind to the estrogen receptor (ER) and exert an estrogenic action. Since activity of isoflavones is so very weak like one-thousandth from ten thousands to one of estrogen, isoflavones also have antiestrogenic effect on female high reproductive age estrogen blood levels. Soybean contains mainly genistin and daidzin as glycosidic isoflavones, and is a valuable functional ingredient that is only 0.2 to 0.3% of the weight of soybean, and the isoflavone content of soybean germ is about compared to that of whole soybean 10 times higher. Moreover, the composition of isoflavones contained in soybean germ is different from the composition of isoflavones contained in whole soybeans, and is rich in daidzin isoflavones. However, glycosidic isoflavones are not absorbed as they are in the body, but are degraded after being degraded to sugar chain of glycosidic isoflavones by enzymes of enteric bacteria and then absorbed into aglycones. Since the aglycone type isoflavone has already been degraded in sugar chains, it is rapidly absorbed by the stomach and intestine without using the function of enteric bacteria (non-patent Literatures 7 to 9).

Previous studies have suggested that oral consumption of isoflavones by in-vitro fertilization patients may improve the effect of progesterone in the luteal phase and may increase the rate of successful embryo transfer and clinical pregnancy rate (Non-patent Literature 10). Plant-derived isoflavones have also been reported to control prostaglandin secretion from the endometrium in the early pregnancy (Non-patent Literature 11). In addition, in the randomized trial of 190 postmenopausal women, AglyMax oral intake for 12 weeks alleviated the hot flush symptom (Non-patent Literature 12). Aglycone type isoflavones are more rapidly absorbed in the body than glycosides and have a higher absorption rate (Non-patent Literature 13). Genistein promotes cell proliferation of estrogen-dependent breast cancer cells (MCF-7) (Non-patent Literature 14) and attenuates the inhibitory effect of tamoxifen (Non-patent Literature 15). On the other hand, daidzein suppressed the proliferation of MCF-7 (Non-patent Literature 16) and improved the therapeutic effect of tamoxifen (Non-patent Literature 17). These reports show that the type of isoflavone has different effects on target cells.

### PRIOR ART LITERATURE

### Patent Literature

Patent Literature 1: Japanese Patent Laid-open Publication No. JP2009-209158A

Patent Literature 2: Japanese Patent Publication No. 3014145

Patent Literature 3: Japanese Patent Publication No. 4397525

### Non-Patent Literature

Non-patent Literature 1: Giudice L C. Clinical practice. Endometriosis. N Engl J Med 2010; 362: 2389-98.

Non-patent Literature 2: Han S J, Jung S Y, Wu S P, Hawkins S M, Park M J, Kyo S, Qin J, Lydon J P, Tsai S Y, Tsai M J, DeMayo F J, O'Malley B W, Estrogen receptor β modulates apoptosis complexes and the inflammasome to drive the pathogenesis of endometriosis. Cell 2015; 163: 960-974.

Non-patent Literature 3: Mori T, Ito F, Matsushima H, Takaoka O, Tanaka Y, Koshiba A, Kusuki I, Kitawaki J. G protein-coupled estrogen receptor 1 agonist G-1 induces cell cycle arrest in the mitotic phase. leading to apoptosis in endometriosis. Fertil Steril 2015; 103: 1228:36.

Non-patent Literature 4: Reis F M, Petraglia F, Taylor R N. Endometriosis: hormone regulation and clinical consequences of chemotaxis and apoptosis. Hum Reprod Update 2013; 19:406-18.

Non-patent Literature 5: Monsivais D, Dyson MT, Yin P, Coon JS, Navarro A, Feng G, Malpani S S, Ono M, Ercan CM, Wei J J, Pavone M E, Su E, Bulun S E. ER β- and prostaglandin E2-regurelated pathways integrate cell proliferation via Ras-like and estrogen-regulated growth inhibitor in endometriosis. Mol Endocrinol 2014; 28:1304-15.

Non-patent Literature 6: Yamanaka K, Xu B, Suganuma I, Kusuki I, Mita S, Shimizu Y, Mizuguchi K, Kitawaki J. Dienogest inhibits aromatase and cyclooxygenase-2 expression and prostaglandin E₂ production in human endometriotic stromal cells in spheroid culture. Fertil Steril 2012; 97:477-82.

Non-patent Literature 7: Chen Y, Chen C, Shi S, Han J, Wang J, Hu J, Liu Y, CaiZ, Yu C. Endometriotic implants regress in rat models treated with puerarin by decreasing estradiol level. Reprod Sci 2011; 18:886-91.

Non-Patent Literature 8: Wagner J D, Anthony M S, Cline J M. Soy phytoestrogens: research on benefits and risks. Clin Obstet Gynecol 2001; 44:843-52.

Non-patent Literature 9: Tsuchiya M, Miura T, Hanaoka T, Iwasaki M, Sasaki H, Tanaka T, Nakao H, Katoh T, Ikenoue T, Kabuto M, Tsugane S. Effect of soy isoflavones on endometriosis: interaction with estrogen receptor 2 gene polymorphism. Epidemiology 2007; 18:402-8.

Non-patent Literature 10: Unfer V, Casini ML, Gerli S, Costabile L, Mignosa M & Di Renzo G C Phytoestrogens may improve the pregnancy rate in vitro fertilization-embryo transfer cycles: a prospective, controlled, randomized trial. Fertility and Sterility 2004; 82:1509-13.

Non-patent literature 11: Woclawek-Potocka I, Bah M M, Korzekwa A, Piskula M K, Wiczkowski W, Depta A, Skarzynski DJ. Soybean-derived phytoestrogens regulate prostaglandin secretion in endometrium during cattle estrous cycle and early pregnancy Exp Biol Med 2005; 203:189-99.

Non-patent Literature 12: Khaodhiar L, Ricciotti HA, Li L, Pan W, Schickel M, Zhou J, Blackburn G L. Daidzein-rich isoflavone aglycones are potentially effective in reducing hot flashes in menopausal women. Menopause 2008; 15:125-32.

Non-patent Literature 13: Izumi T, Piskula MK, Osawa S, Obata A, Tobe K, Saito M, Kataoka S, Kubota Y, Kikuchi M. Soy isoflavone aglycones are absorbed faster and in higher amounts than their glucosides in humans. J Nutr 2000; 130:1695-9.

Non-patent Literature 14: Ju Y H, Allred C D, Allred K F, Karko K L, Doerge D R, Helferich W G. Physiological concentrations of dietary genistein dose-dependently stimulated growth of estrogen-dependent human breast cancer (MCF-7) tumors implanted in athymic nude mice. J Nutr 2001; 131:2957-62.

Non-patent Literature 15: Ju YH, Doerge DR, Allred KF, Allred CD, Heliferich WG. Dietary genistein negates the inhibition effect of tamoxifen on growth of estrogen-dependent human breast cancer (MCF-7) cells implanted in athymic mice. Cancer Res 2002; 62:2474-7.

Non-patent Literature 16: Ju Y H, Fultz J, Allred K F, Doerge D R, Helferich W G. Effects of dietary daidzein and its metabolites, equol, at physiological concentrations on the growth of estrogen-dependent human breast cancer (MCF-7) tumors implanted in ovariectomized athymic mice. Carcinogenesis 2006; 27:856-63.

Non-patent Literature 17: Constantinou A I, White B E, Tonetti D, Yang Y, Liang W, Li W, van Breemen R B. The soy isoflavone daidzein improves the capacity of tamoxifen to prevent mammary tumors. Eur J Cancer 2005; 41:647-54.

Non-patent Literature 18: Saito A, Osuga Y, Yoshino O, Takamura M, Hirata T, Hirota Y, Koga K, Harada M, Takemura Y, Yano T, Taketani Y. TGF- β1 induces proteinase-activated receptor 2 (PAR2) expression in endometriotic stromal cells and stimulates PAR2 activation-induced secretion of IL-6. Hum Reprod 2011; 26:1892-8.

Non-patent Literature 19: Okubo T, Mok S C, Chen S. Regulation of aromatase expression in human ovarian surface epithelial cells. J Clin Endocrinol Metab 2000; 85:4889-99.

Non Patent Literature 20: Takai E, Taniguchi F, Nakamura K, Uegaki T, Iwabe T, Harada T, Parthenolide reduces cell proliferation and prostaglandin estradiol synthesis in human endometriotic stromal cells and inhibits development of endometriosis in the murine model. Fertil Steril 2013; 100:1170-8.

Non-patent Literature 21: Vercellini P, Cortesi I, Crosignani P G. Progestins for symptomatic endometriosis: a critical analysis of the evidence. Fertil Steril 1997; 68:393-401.

Non-patent Literature 22: Bulun S E. Endometriosis. N Engl J Med 2009; 360:268-79.

Non-Patent Literature 23: Kuiper G G, Lemmen J G, Carlsson B, Corton J C, Safe S H, van der Saag P T, van der Burg B, Gustafsson J A. Interaction of estrogenic chemicals and phytoestrogens with estrogen receptor beta. Endocrinology 1998; 139:4252-63.

Non-patent Literature 24: Monsivais D, Dyson MT, Yin P, Navarro A, Coon JS 5th, Pavone ME, Bulun SE. Estrogen receptor β regulates endometriotic cell survival through serum and glucocorticoid-regulated kinase activation. : Fertil Steril 2016; 105:1266-73.

Non-patent Literature 25: Zhang L, Cui R, Cheng X, Du J. Antiapoptotic effect of serum and glucocorticoid-inducible protein kinase is mediated by novel mechanism activating I · B kinase. Cancer Res 2005; 65:457-64.

### DISCLOSURE OF INVENTION

### Problem to be Solved by the Invention

Although isoflavones are drawing attention for women's health care, their effects on endometriosis are unknown, and detailed analysis is needed to understand their clinical effects and pharmacological effects.

Therefore, the present invention has been made in view of the foregoing, and a material for improving endometriosis, which is a material using aglycone type isoflavone and capable of improving endometriosis, and a method for producing the same. It is intended to be provided.

### Means for Solving Problem

In order to achieve the above-mentioned purpose, the present inventors conducted intensive studies, using endometriosis stromal cells isolated from endometriosis cysts and a mouse model of endometriosis, comparing with the effect of aglycone type isoflavone for endometriosis and the effect of glycoside type isoflavone, and finally completed the following material for improving endometriosis of the present invention. As the aglycone type isoflavone, AglyMax (registered trademark) which is a material manufactured by the manufacturing method based on Patent Literature 2 and Patent Literature 3 was used.

The material for improving endometriosis according to the first aspect of the present invention is comprising an aglycone type isoflavone having an effect of improving endometriosis.

And according to such a configuration, endometriosis is ameliorated by administering and applying the aglycone type isoflavone by any method.

In the material for improving endometriosis according to the second aspect of the present invention, in the first aspect,

the action of improving endometriosis is comprising at least one of the following actions;
To suppress proliferation of endometriotic stromal cells,
Reducing gene expression of IL-6, IL-8, aromatase and COX-2 in endometriosis cells,
Decrease the protein level of PGE2 in endometriosis cells,
Reducing aromatase enzyme activity of endometriosis cells,
In endometriosis cells, suppressing the phosphorylation IκB promoted by TNF-α,
Inhibiting nuclear uptake of p65 by fluorescence immunostaining of endometriosis cells,
PHTPP as ERβantagonist suppresses the growth inhibitory effect of aglycone type isoflavone against endometriosis cells,
ERβ knockdown cells counteract the effect of aglycone type isoflavones on endometriosis cells,
MPP as ERαantagonist does not counteract the effect of AglyMax on endometriosis cells to improve endometriosis,
Reducing gene expression of SGK1 in endometriosis cells,
Inhibit cyst formation in endometriosis model mice,
Reducing Ki67 staining rate in endometriosis cells.

And according to such a configuration, many items related to endometriosis are improved by, for example, orally administering the aglycone type isoflavone.

In the method for producing an endometriosis improving material according to the present invention is characterized in that, inoculating legumes with aspergillus oryzae and koji treating them, and hydrolyzing the proteins in the products by hydrolyzing the products of the koji treatment and decomposing glycosides of the isoflavone compounds in the legumes, then producing isoflavone compounds containing a large amount of aglycone, and producing a material for improving endometriosis containing aglycone type isoflavone from the legumes.

And according to such a configuration, it is possible to reliably manufacture an endometriosis improving material, and to reliably provide an endometriosis improving material capable of improving endometriosis. It is possible to exert an excellent effect.

### EFFECT OF THE INVENTION

According to the material for improving endometriosis of the present invention, endometriosis can be improved by a material using aglycone type isoflavone.

According to the method for producing an endometriosis improving material according to the present invention, it is possible to reliably produce an endometriosis improving material using aglycone type isoflavone capable of improving endometriosis.

### BRIEF DESCRIPTION OF DRAWINGS

FIG. 1 A is a characteristic diagram showing proliferation of endometriosis stromal cells and normal endometrium by AglyMax, and B is a characteristic diagram showing growth of endometriotic stromal cells and normal endometrium by isoflavone 40 (described later).

FIG. 2 A is a characteristic diagram showing the state of gene expression for IL-6, IL-8, aromatase and COX-2 by control, AglyMax and isoflavone 40 based on real time PCR, B is a characteristic diagram showing PGE₂ protein level for control and AglyMax, and C is a characteristic diagram showing aromatase enzyme activity for control and AglyMax.

FIG. 3 A is a characteristic diagram showing inhibition of the phosphorylation of IκB promoted by TNF-αfor AglyMax, and B is a characteristic diagram showing inhibition of p65 uptake for AglyMax.

FIG. 4 A is a characteristic diagram showing cell growth suppression of AglyMax and PHTPP, B is a characteristic diagram showing cell growth suppression of AglyMax and MPP, C is a characteristic diagram showing cell growth suppression of AglyMax Against SiERβ and control, D is a characteristic diagram showing SGK1 gene expression of AglyMax and control, and E is a characteristic diagram showing the pharmacological mechanism of AglyMax.

FIG. 5 A is a photograph showing an endometriosis-like lesion, B is a photograph showing the size of A lesion, C is a characteristic diagram showing the number of cysts in mice administered with control, AglyMax and isoflavone 40, D is a characteristic diagram showing cyst weight in the case of C, and E is a characteristic diagram showing the staining ratio of Ki67 in the cyst portion in mice administered with control and AglyMax.

### MODE(S) FOR CARRYING OUT THE INVENTION

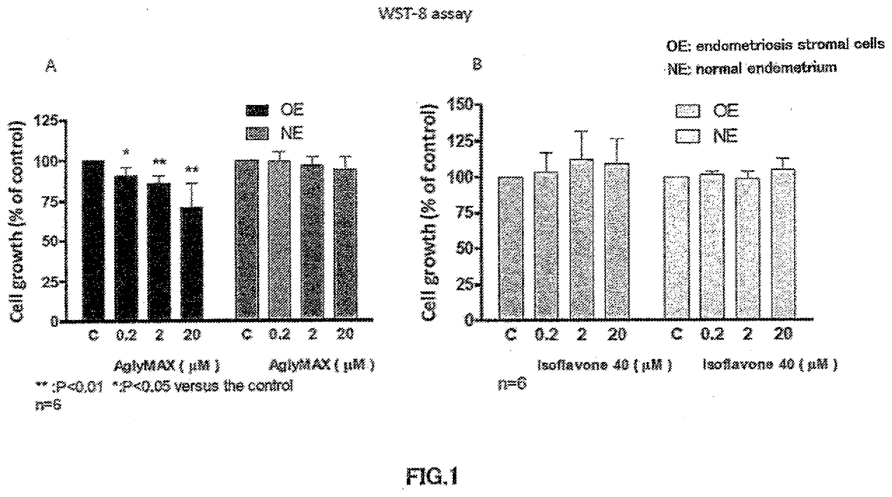

Hereinafter, an embodiment of a material for improving endometriosis according to the present invention will be described with reference to FIGS. 1 to 5.

Based on the present invention, it was confirmed that endometriosis is improved by administering and applying aglycone type isoflavone, which is a material for improving endometriosis, to a specimen by the following experiments and the like.

### <1 Isoflavones>

which is a material for endometriosis improvements of the present invention the AglyMax as aglycone type isoflavone (manufactured Nichimo Biotics Co., Ltd.) was used. The manufacturing method of the raw material for endometriosis improvement is mentioned later. The AglyMax is an aglycone type isoflavone produced by subjecting soybean germ to fermentation with Aspergillus oryzae by the production method based on Patent Literature 2 and Patent Literature 3, and subjected to extraction and concentration processes, and has a composition of daidzein, genistein and glycitein of which ratio is 7:1:2.

As a comparative material, isoflavone 40 (manufactured by Tama Seikagaku Co., Ltd.), which is a precursor of AglyMax and is a glycosidic type isoflavone.

### <2 Specimens>

Patients with 2 specimens, endometriotic stromal cells were obtained from 24 ovarian endometriotic ovarian patients (mean age 36.8 years; 24-44 years). Normal endometrial cells were obtained from 12 patients who underwent ovarian benign tumor removal (mean age 34.9 years; 22-41 years). Samples of patients who did not receive hormone treatment were used at least after 6 months of surgery. The study was started after compliance with the Helsinki Declaration and approval from the Kyoto Prefectural University of Medicine Ethics Review Board (RE-E-306). Informed consent was obtained from all patients.

### <3 Experimental Items>

<3-1 Endometriosis Stromal Proliferation>

(Experimental Method)

The cell treatment and cell proliferation assay as follows were performed.

Endometriotic stromal cells were treated and isolated as described (Non-patent Literature 18). The cells were seeded at 5,000 cells per well in a 96 well plate. Cell proliferation was measured by WST-8 assay after 72 hours of treatment with AglyMax and DMSO for 24 hours. It was measured by a multiwell multiplex 9120 (Bio-Rad) at a wavelength of 450 nm.

(Experimental Results)

AglyMax showed an antiproliferative effect on endometriotic stromal cells, but isoflavone 40 did not. To further illustrate, the effects of AglyMax and isoflavone 40 on endometriotic stromal cells and normal endometrium were measured in the WST-8 assay. Although AglyMax inhibited the proliferation of endometriotic stromal cells in a concentration-dependent manner (P<0.01), it showed no growth inhibitory effect in normal endometrium (see FIG. 1A). On the other hand, isoflavone 40 showed no growth inhibitory effect on endometriosis cells and normal endometrium (see FIG. 1B). Since these results study the AglyMax concentration 20 μM was performed in the later experiments.

<3-2 mRNA and cDNA Synthesis, Real-Time PCR>

(Experimental Method)

In order to perform RNA extraction from endometriotic stromal cells, RNeasy Mini kit (QIAGEN) was used to follow the protocol. RNA concentration was measured by NanoDrop (Thermo Scientific). In order to perform cDNA synthesis, ReverTra Ace qPCR RT Kit (Toyobo) and GeneAmp PCR 9700 (Applied Biosystems) were used. In order to perform Real-time PCR, Power SYBR Green and Step One Real-Time PCR System (Applied Biosystems) were used. The target genes of the primers were IL-6, IL-8, aromatase, COX-2, SGK1 and GAPDH.

(Experimental Results)

AglyMax suppressed the gene expression of IL-6, IL-8, aromatase and COX-2. Isoflavone 40 did not suppress their gene expression. Furthermore, AglyMax suppressed PGE2 protein expression and aromatase enzyme activity.

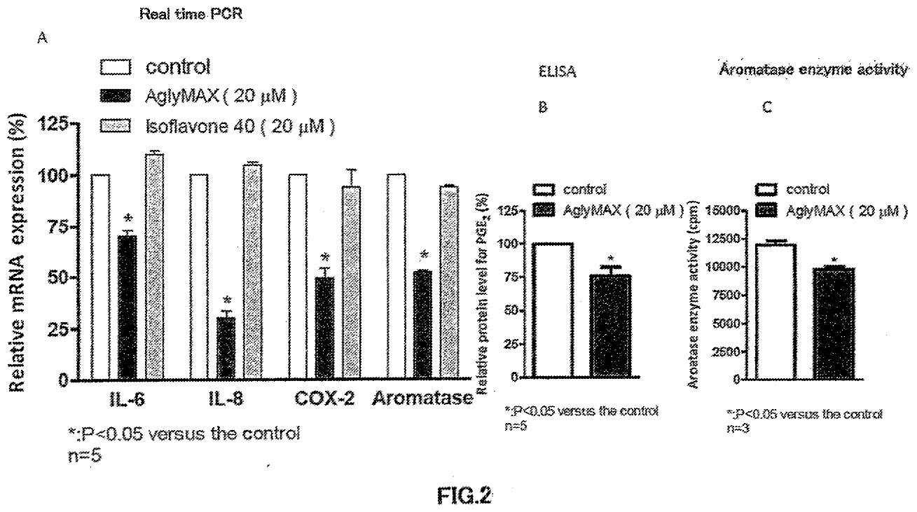

To explain further, gene expression of IL-6, IL-8, aromatase and COX-2 in endometriosis cells was examined by real-time PCR. AglyMax compared with controls 4 reduced the one gene expression (P<0.05) are isoflavones 40 gene expression in was not suppressed (see FIG. 2A). AglyMax reduced PGE₂ protein levels (P<0.05) (see FIG. 2B). Similar to the mRNA level, AglyMax reduced aromatase enzyme activity in endometriosis cells (P<0.05) (see FIG. 2C).

### <3-3 Western Blot and Fluorescent Immunostaining>

(Experimental Method)

In order to perform cell protein extraction, RIPA buffer (Nacalai Tesque) and polyacrylamide gels (ATTO) were used. Phosphorylated inhibitor κB (p-I κB), total-Inhibitor κB (t-I κB), ER · (Abcam), GAPDH (Cell Signaling Technology) were used as primary antibody and against rabbit IgG (Cell Signaling Technology) was used as secondary antibody. After fluorescent immunostaining cells seeded on cover grasse, phosphor-histone H3 (Ser10) was used as primary antibody, and Alexa-conjugated secondary antibodies (Cell Signaling Technology) was used as secondary antibody. The fluorescence was performed by PI (Vector Laboratories) and analyzed with FV1000 confocal laser scanning microscope and FLUOVIEW software (Olympus).

(Experimental Results)

The result of NF-κB route analysis and western blot and immunofluorescent immunostaining were obtained.

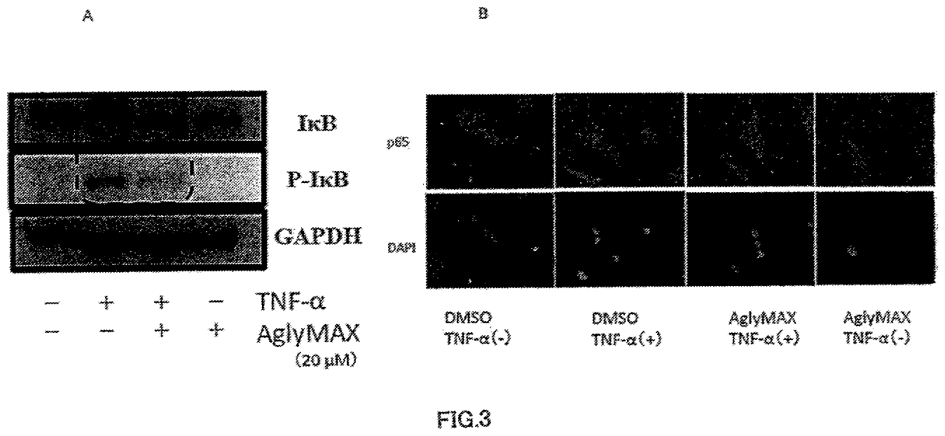

To explain further, the effect of AglyMax on intracellular signaling pathways was examined. AglyMax in endometriotic cells inhibited the phosphorylation of IκB promoted by TNF-α(see FIG. 3A). Furthermore, AglyMax was confirmed to inhibit p65 uptake by fluorescence immunostaining (see FIG. 3B).

### <3-4 ELISA and for the Aromatase Enzyme Activity Measurement>

(Experimental Method)

PGE₂ protein concentration was measured by PGE₂ Highsensitivity enzyme immunoassay (EIA) kit (Enzo Life Science). Aromatase enzyme activity was measured by the previously reported [³H]-water method (Non-patent Literature 19).

(Experimental Results)

AglyMax inhibited cell proliferation of endometriosis through the ERβ. Furthermore, it suppressed the expression of SGK1.

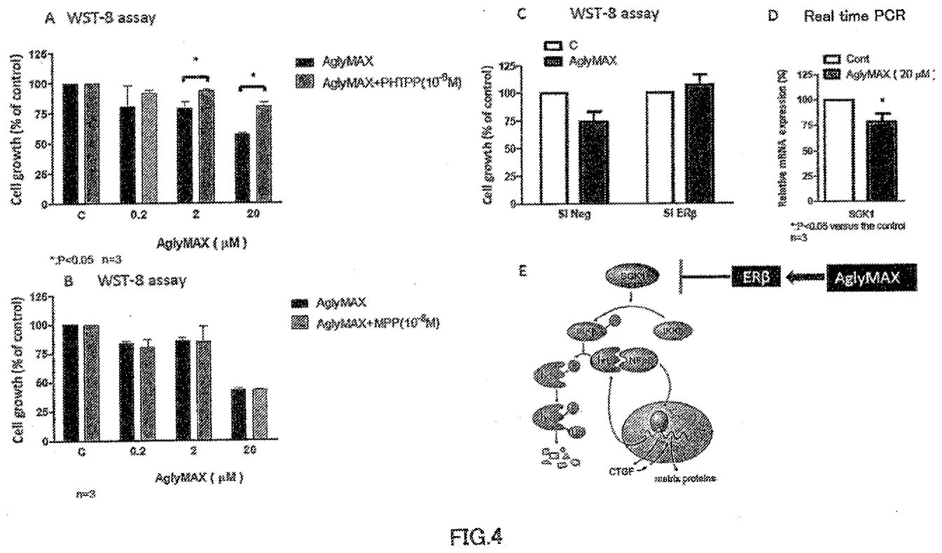

In more detail, the cell growth in the culture solution in which added PHTPP as ERβantagonist was measured by WST-8 assey. PHTPP suppressed the growth inhibitory effect of AglyMax (see FIG. 4A). Further after ERβ knocked down by transferring siRNA, the growth inhibitory effect were examined by WST-8 assay. ERβ knockdown cells negated the effect of AglyMax (see FIG. 4B). MPP as ERα antagonist was added and similarly measured in the WST-8 assay, but the effect of AglyMax was not counteracted (see FIG. 4C). Furthermore, AglyMax reduced SGK1 gene expression (P<0.05) (see FIG. 4D).

### <3-5 Examination by Endometriosis Model Mouse>

(Experimental Method)

Estradiol (E2:0.5 μg/mouse/week) is administered after hysterectomy of BALB/c mice (7 weeks old) to stabilize hormone dynamics Uterine tissue extracted from the same type mice was shredded and administered intraperitoneally (Non-patent Literature 20). One week after uterine tissue transplantation, a feed containing 0.6% and 0.65% of AglyMax and isoflavone 40, respectively, was given. Four weeks after bait administration, cysts formed in the abdominal cavity were removed. The number of cysts and the weight of cysts were measured. Cell proliferation was assessed by Ki67 staining

(Experimental Results)

AglyMax inhibited cyst formation in endometriosis model mice, but isoflavone 40 did not show any suppressive effect.

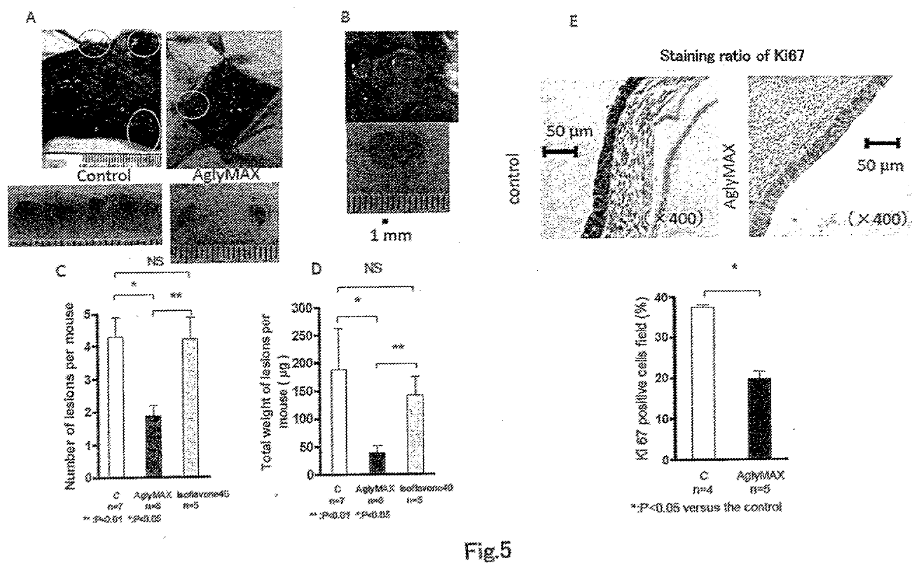

To explain further, endometriosis-like lesions engrafted in the abdominal cavity of mice (see FIG. 5A). Lesion size was 2 to 10 mm (see FIG. 5B). The cyst counts (see FIG. 5C) and cyst weights (see FIG. 5D) of mice fed with AglyMax were reduced (P<0.05) compared to control and isoflavone 40. AglyMax reduced the Ki67 staining rate compared to controls (P<0.05) (see FIG. 5E).

### <3-6 Statistical Analysis>

Statistical analysis was performed using the Mann-Whitney U-test, and data were shown as mean ±standard error. P value made 0.05 or less significant difference.

### <4 Summary of Effects>

This experiment is the first study to investigate the drug effects of endometriotic stromal cells and normal endometrial cells, AglyMax and isoflavone 40 on endometriosis model mice. Since AglyMax has an antiproliferative effect only on endometriosis cells, it is suggested that it can be used for endometriosis patients. Furthermore, it is confirmed that pharmacological actions exhibit via Erβ path and -NF-κB path. AglyMax inhibited cyst formation in model mice but not in isoflavone 40. Concentration of AglyMax was studied under 20 μM, but more detailed density setting is necessary.

Endometriosis is a local pelvic inflammatory disease (Non-Patent Literature 21). In endometriosis tissue, active macrophages secrete inflammatory cytokines such as IL-6 and IL-8. These cytokines contribute to the maintenance of the development of the lesion. AglyMax suppressed the mRNA expression of IL-6 and IL-8. This result is involved in cell growth suppression. Furthermore, suppression of aromatase, COX-2 and PGE₂ by AglyMax suggests that it is effective in suppressing local estrogen production and pelvic pain. To summarize, AglyMax suppressed endometriosis exacerbation related factors.

Here, we analyze how AglyMax exerts anti-inflammatory effects and cell growth suppression on endometrial cells. Endometriosis cells have higher expression of ERβ as compared to normal endometrium (Non-patent Literature 22). Daidzein as a main configuration isoflavones of AglyMax binds weakly to Erβ (Non-Patent Literature 23). As shown in the results, blocking ERβ reduced the effect of AglyMax. Since the effect of AglyMax was not suppressed in ERα, it was shown that the pharmacological effect was mediated by ERβ.

NF-κB is an important control factor in endometriosis. AglyMax inhibited IκB phosphorylation facilitated by TNF-αand nuclear uptake of p65, it showed anti-inflammatory activity. In endometriosis SGK1 is controlled by ERβ (Non-Patent Literature 24). SGK1 promotes IκB phosphorylation and NF-κB activity (Non-Patent Literature 25). AglyMax was also suppressed gene expression of SGK1. AglyMax exerts an anti-inflammatory effect by suppressing this pathway (see FIG. 5E).

Evaluation of the effects of AglyMax and isoflavone 40 was evaluated in endometriosis model mice. One week after uterine tissue transplantation, a feed containing 6% and 6.5% of AglyMax and isoflavone 40, respectively, was given. AglyMax reduced cyst count and cyst weight compared to control and isoflavone 40. These results demonstrate that even when AglyMax is absorbed into the mouse body, it has the same effect as in vitro.

In conclusion, AglyMax inhibited cell proliferation of endometriosis, inhibited pathogenesis of endometriosis model mice via Erβpath and -NF-κB path. These pharmacological effects are expected to suppress the development of lesions of endometriosis and pelvic pain.

### <5. Manufacturing Method of Material for Endometriosis Improvement>

<5-1 Manufacturing Process>

The method of manufacturing the material for endometriosis improvement may be performed by the method described in Patent Literature 2 and Patent Literature 3.

Specifically, it is performed as follows.

That is, in Patent Literature 2, beans are inoculated with Aspergillus oryzae and made into koji mold, and by hydrolyzing the product in the product by hydrolyzing the product from the koji treatment, distribution of the isoflavone compound in the beans is also performed. Glycol is decomposed to produce an isoflavone compound containing a large amount of aglycone, thereby producing a material for improving endometriosis using the beans as a raw material.

In this case, simultaneously removing phytic acid in the beans is also performed.

Further, in Patent Literature 3,
(1) a step of degreasing soybean hydrolyzed by fermentation using a microorganism with a nonpolar solvent, and drying the obtained defatted product, and
(2) the dried defatted product obtained in step (1) is extracted with a polar solvent, and the extract containing the obtained isoflavone compound is concentrated to dryness, and
(3) the concentrated dried extract obtained in step (2) is dissolved in a polar solvent and further hydrolyzed and separated to obtain an insoluble substance, and the polar solvent is methanol, ethanol, propanol, acetone, or a mixture of these with water, and
(4) the insoluble material obtained in step (3) is washed if necessary, dried, and the step of removing the solvent is carried out to produce a material for improving endometriosis. ps <5-2 Raw Material>

In the example of Patent Literature 2, soybean meal in beans is used as a raw material.

In the example of Patent Literature 3, soybean hypocotyl in soybean is used as a raw material.

As a raw material of endometriosis improving material, it is preferable to use soybean hypocotyl containing agricon type isoflavone in a large amount.

<5-3 Material for improving Endometriosis as a Product Result>

The material for improving endometriosis produced by the manufacturing methods described in Patent Literature 2 and Patent Literature 3 using soybean germ as a raw material contains a large amount of aglycone type isoflavone contains.

The aglycone type isoflavones include daidzein, genistein and glycitein. Even one of these can be used as a material for improving endometriosis.

In particular, in AglyMax, daidzein, genistein and glycitein are at a weight ratio of 7:1:2.

Thus, according to the producing method of the present invention, an endometriosis-improving material can be reliably produced, and an endometriosis-improving material capable of improving endometriosis is surely provided. It is possible to exert an excellent effect.

1. A method for improving endometriosis, comprising administrating a composition containing aglycon type isoflavones and a pharmaceutically acceptable carrier to mammal subject in need of treatment of endometriosis in an amount effective for treating endometriosis, wherein the aglycon type isoflavones are composed of daidzein, genistein, and glycitein.

2. The method according to claim 1, wherein the aglycon type isoflavones contain the daidzein, genistein, and glycitein approximately at a weight ratio of 7/1/2.

3. The method according to claim 1, wherein the aglycon type isoflavones consist of the daidzein, genistein, and glycitein.

4. The method according to claim 1, wherein the composition contains the aglycon type isoflavones at a concentration of about 20 μM.

5. The method according to claim 1, wherein the aglycon type isoflavones are derived from soybeans.

6. The method according to claim 1, wherein the aglycone-type isoflavones have the following properties at a concentration of about 20 μM, which are at least partially mediated via suppression of tumor necrosis factor (TNF)-α induced IκB phosphorylation, and inhibition of p65 uptake in nuclei of endometriotic stromal cells.

7. The method according to claim 6, wherein the properties include:
suppressing cell proliferation of endometriotic stromal cells,
reducing gene expressions of interleukin (IL)-6, IL-8, aromatase and cyclooxygenase-2 (COX-2) in endometriosis cells,
reducing the protein level of prostaglandin (PG) E2 in endometriosis cells,
reducing aromatase enzyme activity of endometriosis cells,
PHTPP as Erβ antagonist suppresses the growth inhibitory effect of aglycone type isoflavone against endometriosis cells compared to the case without the aglycone-type isoflavone,
reducing gene expression of serum glucocorticoid regulated kinase (SGK)1, a transcriptional target of estrogen receptor (ER)β, in endometriosis cells,
reducing the number and weight of the lesions in endometriosis model mice, and
reducing the staining rate of Ki-67, a cell proliferation marker, in endometriosis cells.

8. A method for producing a material for improving endometriosis, comprising steps of:
(i) inoculating legumes with aspergillus oryzae and koji treating them; and
(ii) hydrolyzing proteins of a product obtained in step (i) by hydrolyzing the product and decomposing glycosides of isoflavone compounds contained in the legumes, thereby producing isoflavone compounds containing aglycone as the material for improving endometriosis containing aglycone type isoflavone from the legumes.
